(19) United States
(12) Patent Application Publication
(10) Pub. No.: US 2007/0083235 A1
(43) Pub. Date: Apr. 12, 2007

(54) Helical retainer, tool for using the helical retainer, and methods

(76) Inventors: James E. Jervis, Atherton, CA (US); John Ashley, San Francisco, CA (US)

Correspondence Address: WILSON SONSINI GOODRICH & ROSATI, 650 PAGE MILL ROAD, PALO ALTO, CA, 94304-1050, US

(21) Appl. No.: 11/249,008

(22) Filed: Oct. 11, 2005

(51) Int. Cl. A61B 17/04

(52) U.S. Cl. 606/232

(57) ABSTRACT

The invention relates to helical retainers, particularly helical retainers adapted for use in laparoscopic surgery. One aspect of the invention provides a helical retainer having a sterile coiled body adapted and configured to secure a surgical suture, the coiled body having a plurality of coils and being formed from a strand and an extension of the strand extending from the coiled body adapted to engage a suture or to be grasped by a holding tool. Another aspect of the invention provides a method of using a helical retainer including the steps of placing a helical retainer at a desired location, the helical retainer comprising a plurality of coils, engaging a suture with the helical retainer at a first engagement point, and engaging the suture with the helical retainer at a second engagement point to secure the suture to the helical retainer.

### BACKGROUND OF THE INVENTION

(a) Field of the Invention

This invention relates generally to helical retainers, methods of using helical retainers, and tools adapted for use with the helical retainers of the invention. The helical retainers can be used with sutures, thread, wire, string, rope, filament, twine, line, cordage, cable, etc. The helical retainers are formed from a strand or rod made in a suitable length and having a suitable diameter that is capable of being wound into a helical configuration and adapted for a particular use. The strand or rod can be made of any suitable material that provides strength and enables the strand or rod, once wound into a helical shape, to retain its shape. Suitable materials include metals and polymers. In particular, the invention relates to a helical suture retainer adapted for use with surgical sutures applied to a surgical patient, and for use during laparoscopic and other minimally invasive surgical procedures.

(b) Description of the Related Art

Sutures are used for a variety of surgical purposes, such as approximation of tissue and ligation of tissue. Most generally, when placing sutures, the strand of suture material to be used has a needle affixed to one end. The needle is passed through the tissue to be approximated or ligated, forming a stitch. The stitch is then tensioned appropriately, and the two ends of the suture, the needle end and the non-needle end, are knotted to retain the desired tension in the stitch. Sometimes the ends are knotted together; in other instances they may be stoppered separately, often with the aid of a clip, retainer or other device to form resistance in order to prevent passage through tissue. While forming knots in a suture during open surgery is a simple matter, forming knots in sutures during endoscopic surgery can be very difficult. For example, placing sutures during a laparoscopic procedure can require two surgeons to cooperate in a multi-step process which is performed with multiple instruments to pass the needle and suture back and forth to tie the suture knot.

In laparoscopic suturing, although much of the knotting is the same many of the manual degrees of freedom available in open surgery are lost, thus making the process more difficult. Tools, called “graspers,” resembling miniature pliers on the ends of long, tubular extensions must operate through fixed points of entry to access the suturing site. Manipulating sutures in this environment requires new forms of dexterity based solely on video feedback which generally fails to provide depth perception and thus is more difficult. Yet the degree of precision required in instrument positioning is no less than in open surgery.

Suture retainers may be used in place of suture knots to prevent passage of a suture end into and through tissue and to maintain the tension applied to the suture material during the suturing procedure and the therapeutic process. Suture clips and retainers are described in the following publications: U.S. Pat. Nos. 5,234,449; 5,383,905; 5,391,173; 5,643,295; 5,645,553; 5,735,877; 5,845,645; 5,895,393; 5,948,001; 6,039,176; 6,896,686; 6,015,428; 6,066,160; 6,231,592; and 6,432,123; and US Patent Pub No. US2004/0260344 A1.

Current retainer solutions known in the art can present a variety of problems for the user, including limitations with respect to suture size and ease of placement as well as accuracy of placement and ability to adjust the suture tension. What is needed is a device that can be used in cooperation with the suture to provide knot functionality while simplifying the suture handling steps, particularly suture handling during laparoscopic surgery.

### SUMMARY OF THE INVENTION

One aspect of the invention provides a helical retainer including: a sterile coiled body adapted and configured to secure a surgical suture, the coiled body comprising a plurality of coils and being formed from a strand; and an extension of the strand extending from the coiled body adapted to engage a suture or to be grasped by a holding tool. The extension may be coiled, and the extension may extend tangentially from the coiled body. In some embodiments, the plurality of coils of the coiled body form a lumen and a first end of the strand is terminated within the lumen. Some embodiments include a second coiled body joined to the first coiled body.

In some embodiments of the invention, the coils are pre-tensioned. In other embodiments, the helical retainer is preloaded with a suture. In still other embodiments, the helical coil is further adapted to permit a secured suture to be disengaged from the helical retainer without damaging the suture.

In some embodiments, the helical retainer includes one or more gaps between adjacent coils. In other embodiments, the coiled body is further adapted to be engaged by a surgical suture between at least two pairs of adjacent coils to secure the surgical suture.

Another aspect of the invention provides a helical retainer including: a sterile coiled body adapted and configured to secure a surgical suture, the coiled body comprising a plurality of coils; and a gap between two adjacent coils. In some embodiments, the helical coil is further adapted to permit a secured suture to be disengaged from the helical retainer without damaging the suture. In other embodiments, the coiled body is further adapted to be engaged by a surgical suture between at least two pairs of adjacent coils to secure the surgical suture.

Yet another aspect of the invention provides a method of using a helical retainer including the following steps: placing a helical retainer at a desired location, the helical retainer comprising a plurality of coils; engaging a suture with the helical retainer at a first engagement point; and engaging the suture with the helical retainer at a second engagement point to secure the suture to the helical retainer to, e.g., apply tension to urge one coil of the helical retainer toward another coil of the helical retainer to more tightly engage the suture between the coils. In some embodiments, the step of engaging the suture at the first engagement point includes the step of engaging the suture at the second engagement point and advancing the suture along the helical element from the second engagement point toward the first engagement point. In some embodiments, the first and second engagement points are between adjacent pairs of coils. Some embodiments include the step of releasing the suture from the helical retainer. Other embodiments include the step of applying tension to the suture by, e.g., pulling an end of the suture. Still other embodiments include the step of engaging the suture with the helical retainer a second time at the second engagement point.

Another aspect of the invention provides a kit for suturing including a sterile coiled body adapted and configured to secure a surgical suture, the coiled body having a plurality of coils and being formed from a strand and an extension of the strand extending from the coiled body adapted to engage a suture or to be grasped by a holding tool; and a suture secured by the helical body.

Yet another aspect of the invention provides a tool adapted to engage a suture with a helical retainer including: a proximal end having a handle; and a distal end adapted to guide a suture into engagement with the helical retainer. In some embodiments, the distal end is retractable with respect to the proximal end.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A is perspective view of a helical retainer having a single tangential wire extending therefrom; FIG. 1B is a perspective view of another helical retainer having a tangential wire extending from a first end and a second end; FIG. 1C is a side view of another helical retainer having a gap formed between an outer coil and an adjacent coil at one end; FIG. 1D is a side view of yet another helical coil having a gap formed between an outer coil and an adjacent coil at both ends;

FIGS. 2A-B are side views of helical retainers, such as those shown in FIG. 1; FIG. 2A illustrates a helical retainer having a left handed turn, or helix; FIG. 2B illustrates a helical retainer having a right handed turn, or helix;

FIGS. 3A-F are side views of a helical retainer of the invention illustrating a process of anchoring a suture with the helical retainer;

FIGS. 4A-E are side views of a helical retainer of the invention illustrating a process of anchoring two suture legs within the helical retainer;

FIGS. 5A-I are side views of a helical retainer of the invention illustrating another process of anchoring a suture in conjunction with the helical retainer;

FIGS. 6A-F are side views of a helical retainer of the invention illustrating yet another process of anchoring a suture in conjunction with the helical retainer;

FIG. 7A is a perspective view of a helical retainer and grasping device with an extensible element, showing a suture being threaded to be secured thereto; FIG. 7B is a perspective view of a helical retainer and alternate extensible element, showing a suture being threaded on a suture retainer to be secured to the suture retainer;

FIGS. 8A-C are perspective views of alternate designs of the helical retainer; FIGS. 8D-E are top and side views of another alternate design in which the end extension turns inwards into the interior of the coiled wire form;

FIGS. 9A-C are top views of alternate circumferential geometries suitable for use with the helical retainer of the invention;

FIG. 10 illustrates the helical retainer of FIG. 1B in side view with a suture secured at each end;

FIGS. 11A-M illustrate top and side views of various extension designs suitable for use with the helical retainer of the invention; and

FIG. 12 illustrates a an alternative helical retainer and suture configuration.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the claims define the scope of the invention and that methods and structures within the scope of these claims and their equivalents be covered thereby.

This invention comprises helical retainers used to secure a length, or lengths, of suture. The suture can be secured along its length at one or both legs or ends. In most instances, the helical retainer, or helical retainer in combination with the suture, or other elements, forms a stop on the leg or legs of suture, preventing passage of the suture through tissue, i.e., providing a stopper or anchor. As will be appreciated by those skilled in the art, the stopper or anchor can be achieved by using configurations of the suture relative to the helical retainer that provide resistance to the movement of the suture relative to the retainer.

The helical retainer is used in combination with a suture, i.e., the fine thread or other material used surgically to close a wound or join tissues. Suitable suture materials are known in the art. The helical retainer can also be used with a tool for manipulating the retainer and the suture relative to the retainer, as described below. As will be appreciated, however, the helical retainer devices of the invention can be adapted to engage a variety of other materials and function in a variety of other environments without departing from the scope of the invention. For example, the helical retainers can be adapted for use with thread, wire, string, rope, filament, twine, line, cordage, cable, etc.

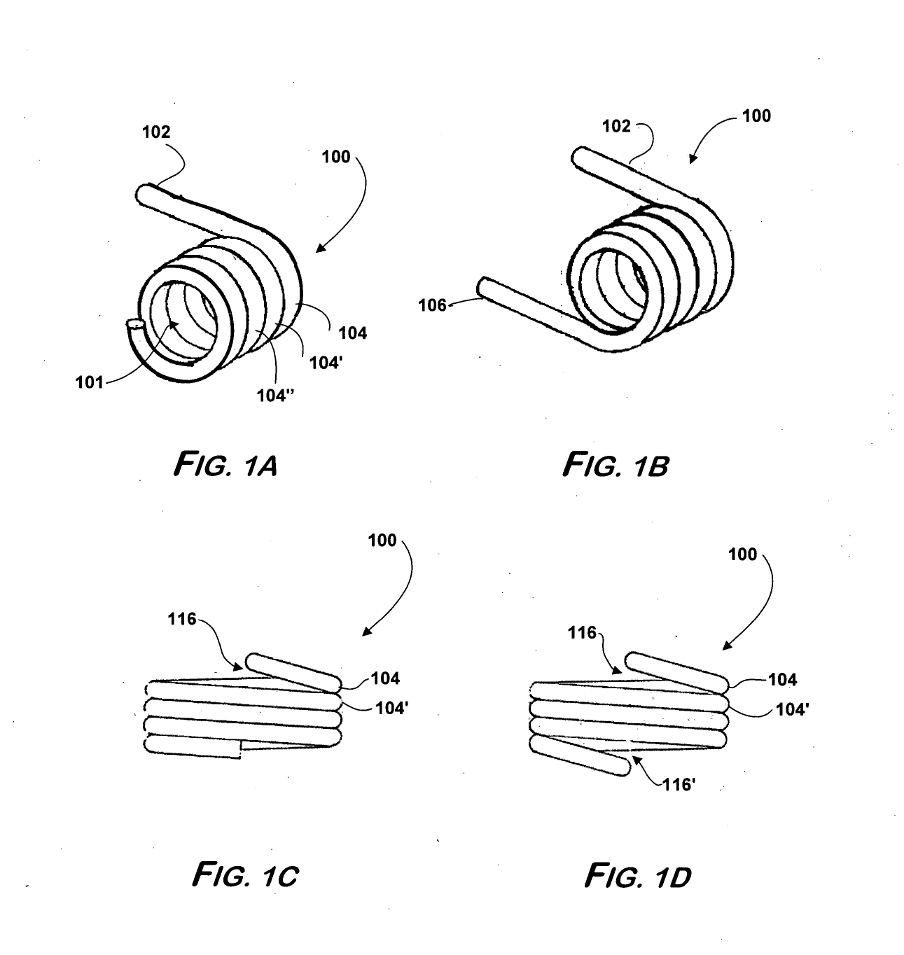

A somewhat generic helical retainer 100, or spring, is shown in FIG. 1A. The helical retainer 100 is formed from a strand or rod made in a suitable length and having a suitable diameter that is capable of being wound into a helical configuration. The strand can include a metallic strand or wire. Once wound into a helical configuration, the helical retainer defines a lumen 101 within the coils 104, 104′, 104″. The strand or rod can be made of any suitable material known in the art that provides strength and enables it to retain its shape. Materials include metals, such as stainless steel, nitinol, and titanium and polymers such as polyester, polycarbonate, polyamide, and bioresorbable polymers. The size of the helical retainer 100 can be adapted for performance in a variety of situations where anchoring is desired. Further, the strand or rod can be formed of multiple individual elements and need not be a singular strand. For purposes of illustration, the helical retainer 100 has been described below in the context of use with surgical sutures within a human body because it illustrates many of the desirable features and functionality of the device design. Other uses and contexts will become apparent based on the teachings of this disclosure.

The helical retainer 100 is a simple helix with an extension 102 at one end. The extension 102 can extend tangentially to the geometric shape of the helical retainer 100, as illustrated, or can achieve another orientation without departing from the scope of the invention. The helical retainer 100 includes a plurality of coils 104, 104′, 104″. The number of coils provided can vary depending upon the desired length and/or function of the helical retainer 100 and typically includes two coils or more. The coils define a lumen 101 within the interior diameter of the coils. The shape of the coils can be configured to define a variety of geometric shapes.

As shown in FIG. 1B the helical retainer 100 can have a second tangential extension 106. Such a configuration might be useful where, for example, a portion of a suture was to be secured to each end of the helical retainer 100. The tangential extensions 102, 106 can be used to grasp the helical retainer during use, as discussed in further detail below. The extension 102 provides a guide for engaging the suture with the helical retainer such that the helical retainer 100 does not need to be preloaded since the suture can be easily led adjacent the extension 102 and fed between the extension and a target set of coils 104 and 104′ using the extension as a guide. The extensions 102, 106 can be configured such that the extensions are parallel with respect to each other, although not on the same side of the retainer, as illustrated, or at an angle relative to the other. Configuration changes may be made to promote stability of the device within a specific environment to optimize control. It is not necessary that the helical retainer comprise an extension as described above.

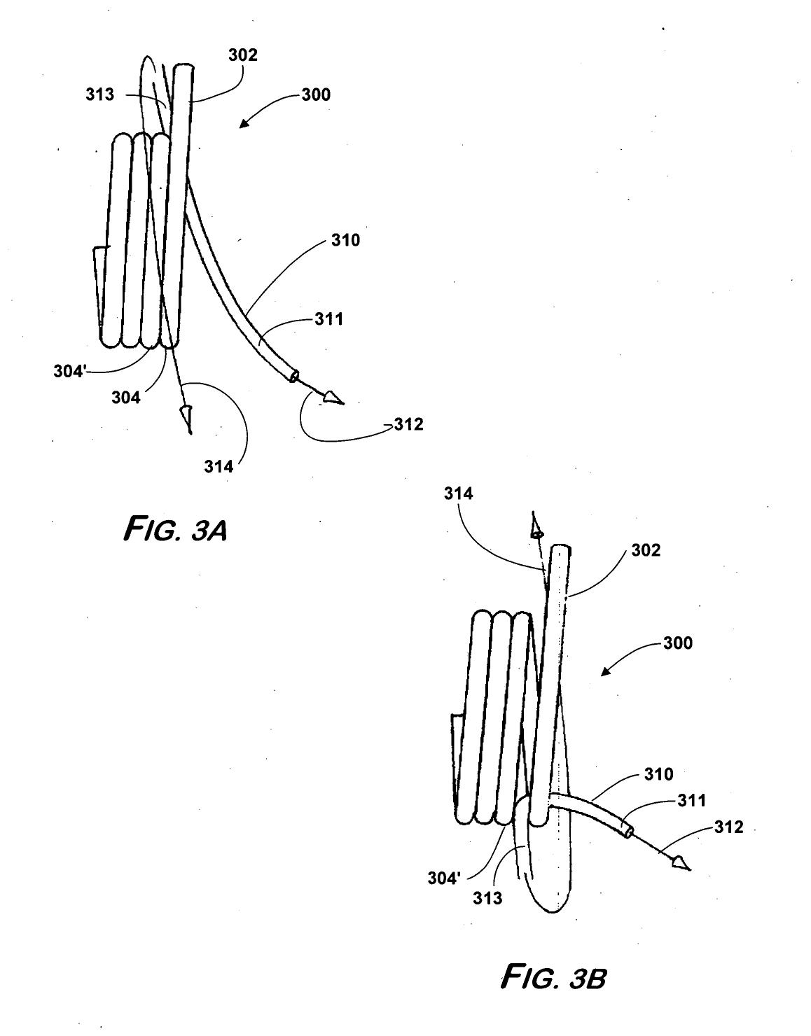

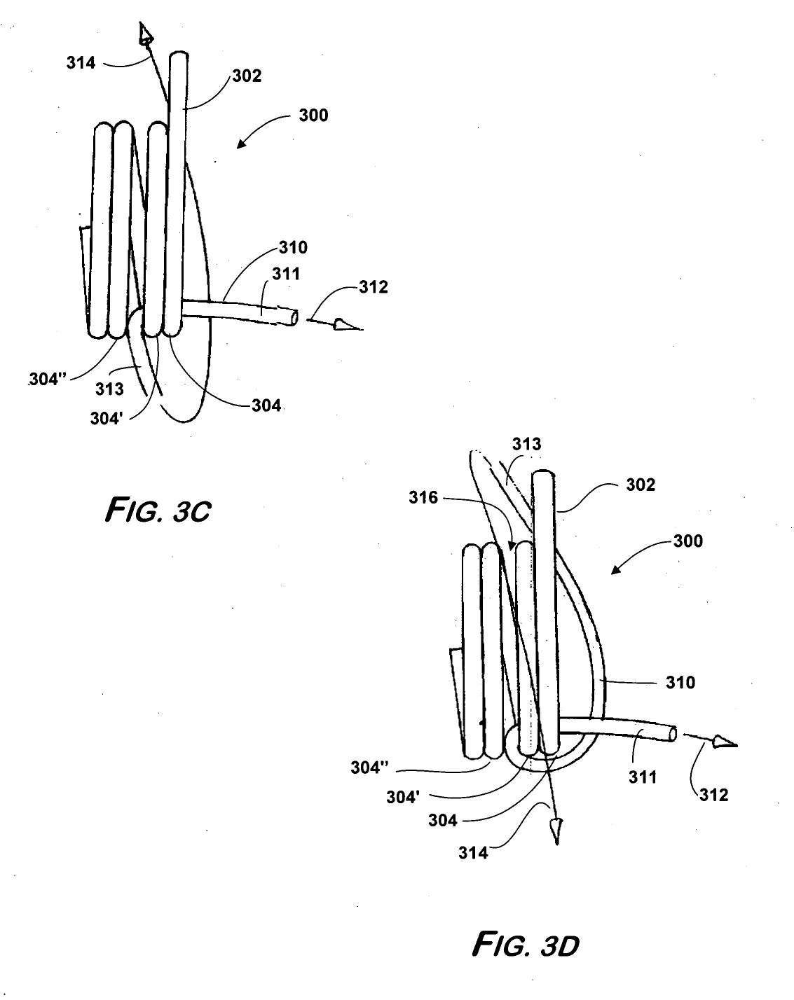

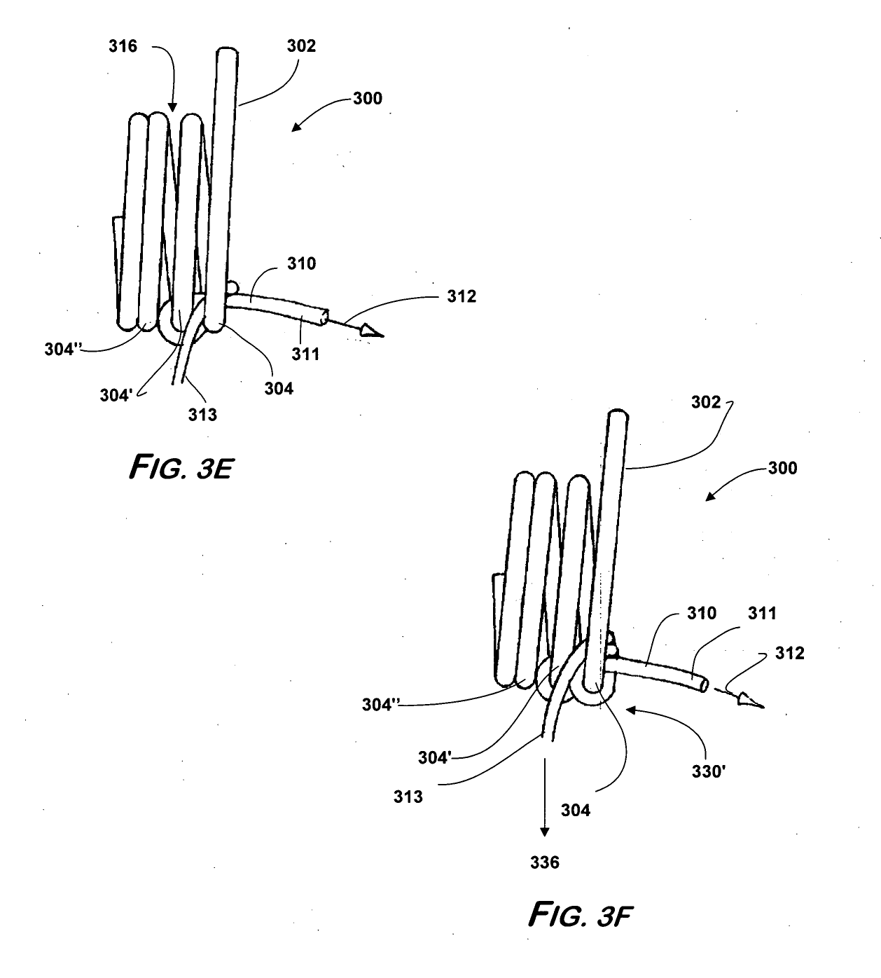

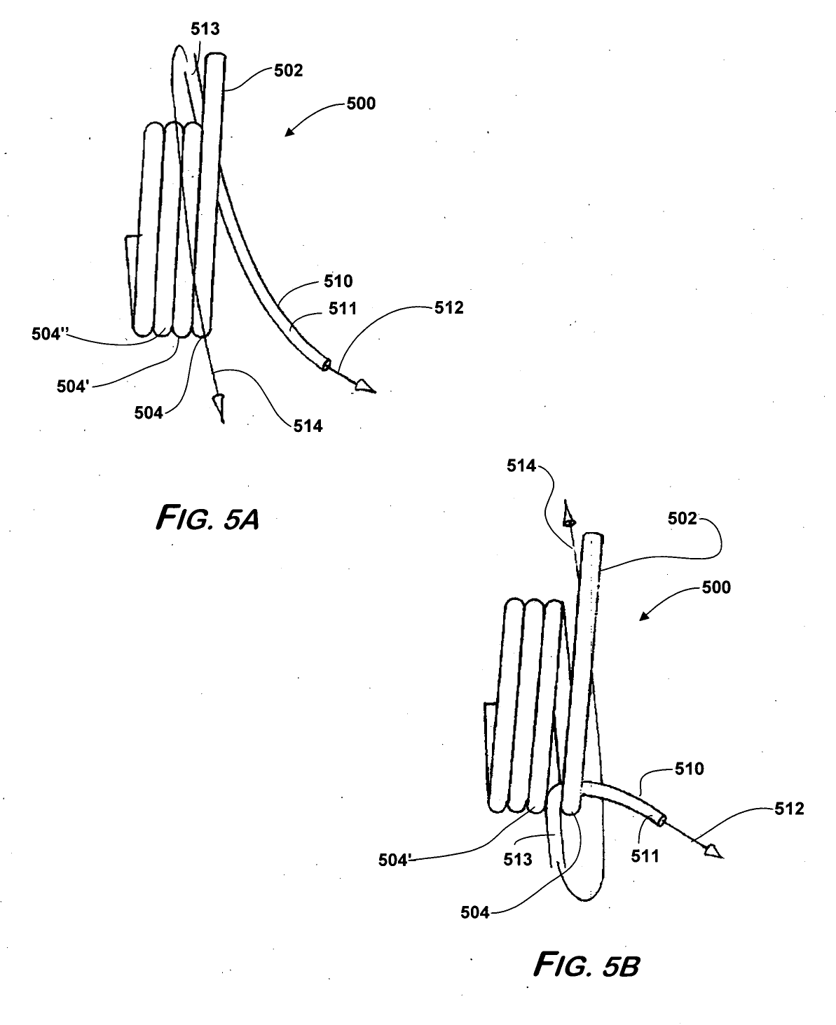

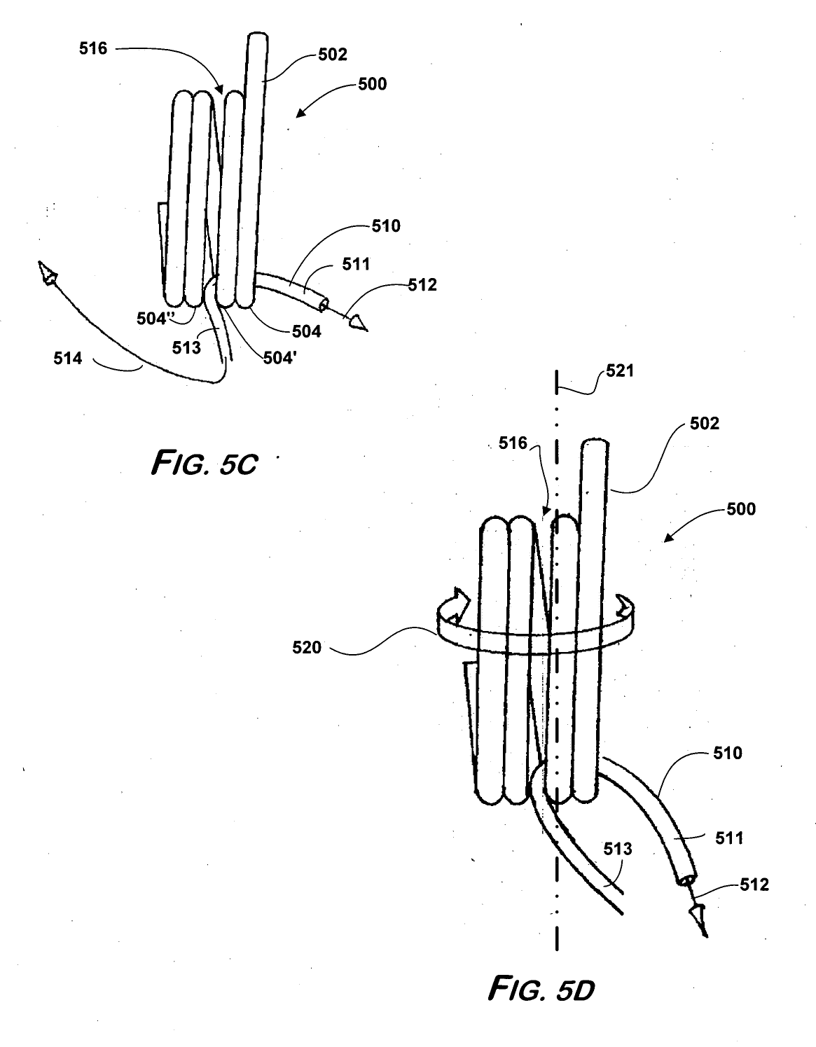

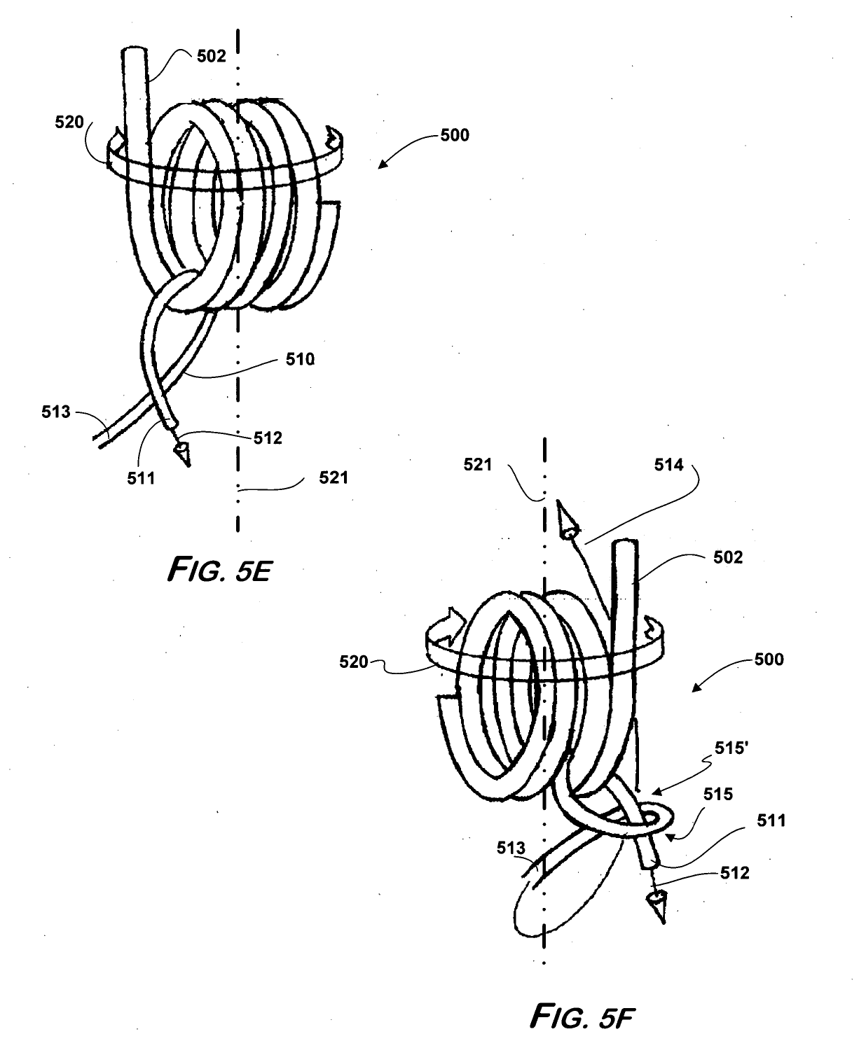

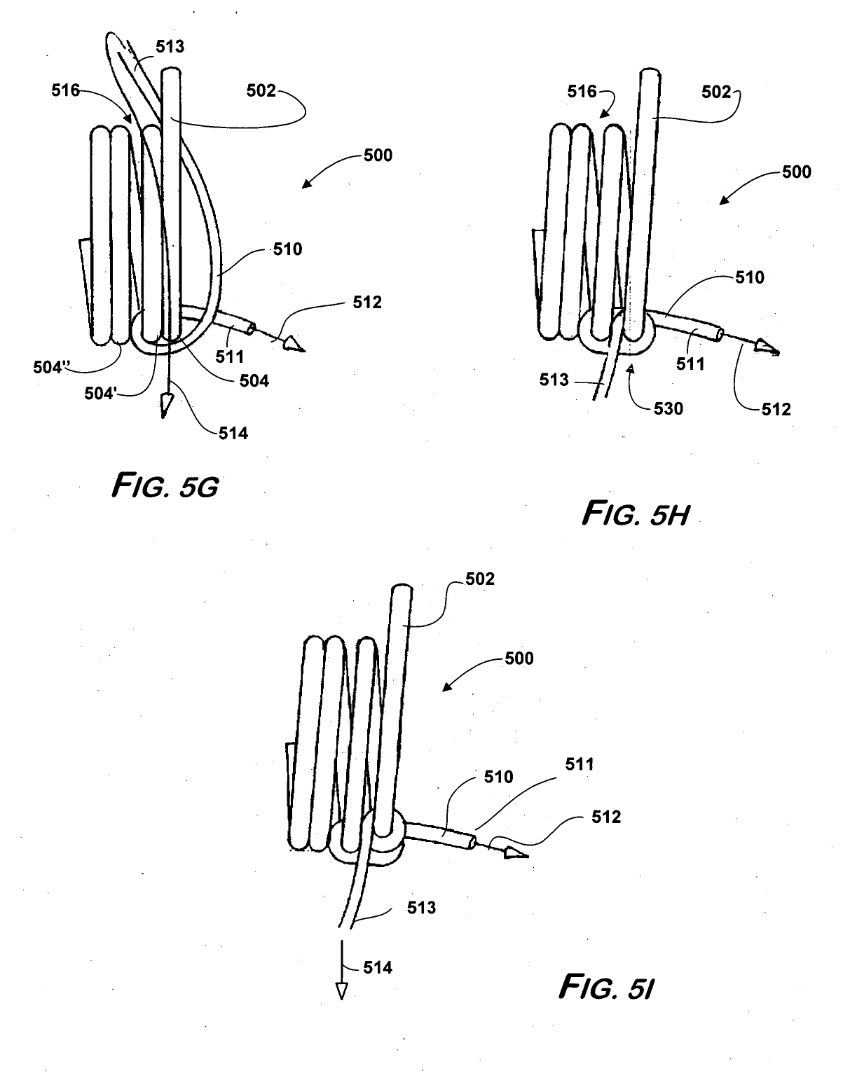

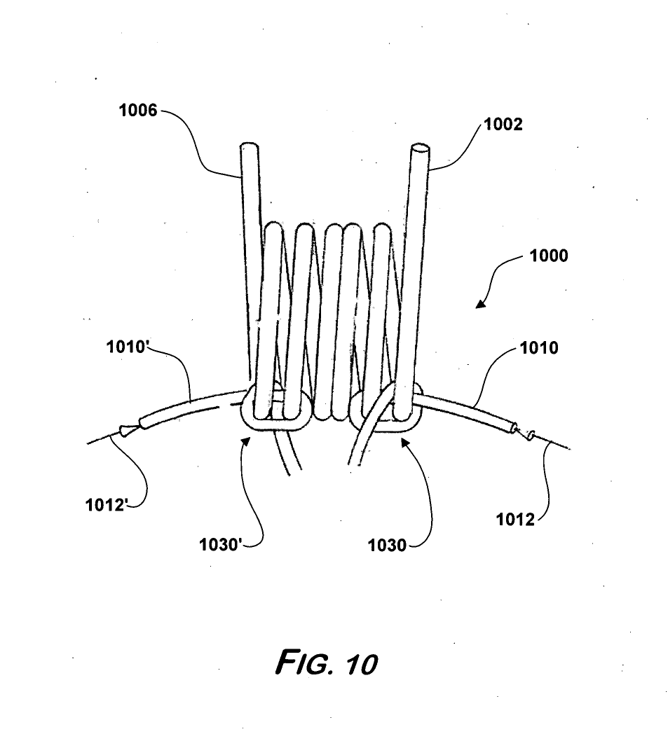

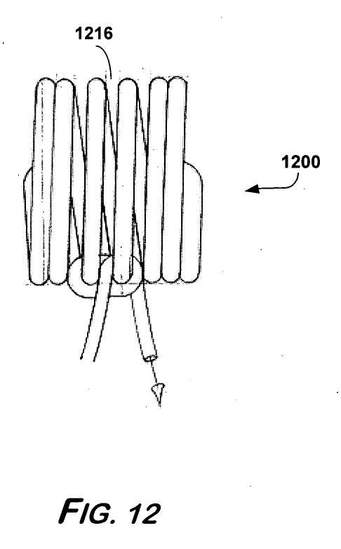

FIG. 1C shows a helical retainer 100 wherein a gap 116 is formed between the end coil 104 and the adjacent coil 104′ of the helical retainer 100 at one end to facilitate feeding suture between the coils of the helical retainer 100. FIG. 1D shows a helical retainer similar to that in FIG. 1C but with gaps 116, 116′ formed in each end. Helical retainers like those of FIGS. 1B and 1D can be used to terminate two sutures sequentially, for example, one suture leg at each end. FIG. 10, discussed below, shows such a situation utilizing exemplary anchors of FIGS. 3A-D. In other embodiments, gaps may be formed between any or all pairs of adjacent coils. For example, FIG. 12 shows an embodiment wherein a gap 1216 is formed between a pair of adjacent coils toward the center of the helical retainer 1200. The suture anchoring element of FIG. 12 is formed using a process similar to that explained below with respect to FIG. 5.

Pre-tensioning is a common feature of conventional extension springs. With extension springs, such as those depicted in FIGS. 1A-D, some or all of the coils comprising the helical retainer can be pre-tensioned such that the coils of the spring, when not engaged by a suture, are immediately adjacent and forcibly pressed against one another. One result of this feature is that a positive force is necessary to initiate extension of the helical retainer. Pre-tensioning the helical retainer 100 can be helpful in holding the suture in place during various steps of anchor formation. Further, pre-tensioning may assist in tensioning of the suture before the anchor formation is complete. The helical retainer 100 can be incorporated into other apparatus as a suture terminating element, such as bone anchors or retaining mesh or can have elements added to it such as a rounded, molded plastic element or formed metal at the coil ends to minimize tissue trauma.

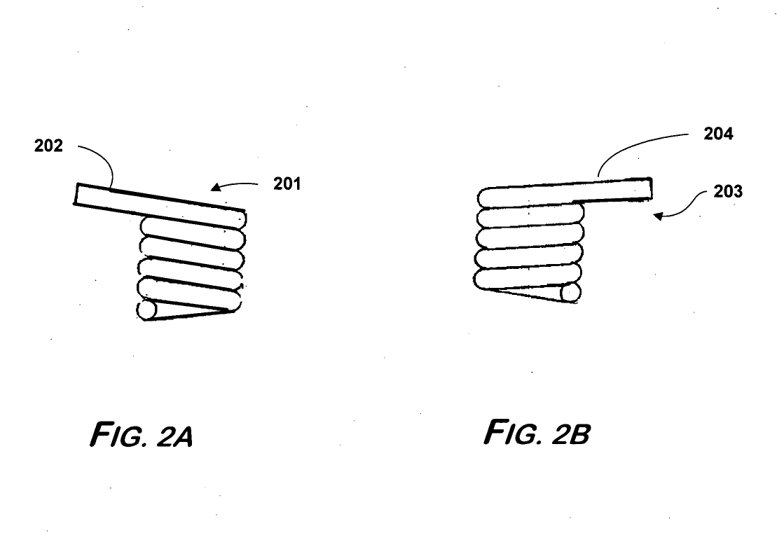

FIG. 2A illustrates a helical spring 201 like an extension spring having a left-handed helix with the extension 202 being positioned proximal and to the left; FIG. 2B illustrates a helical spring 203 having a right handed helix with the extension 204 being positioned proximal and to the right As will be apparent in the description below of the advancement of the suture relative to the retainer, the selection of either a left-handed or right-handed helix affects the direction of this advancement but in no way limits the retaining function of the device

Embodiments of the invention can include an extension spring element, preferably with some or all coils pre-tensioned, and can further comprise extensions, or gaps, helical or non-helical, at one or both ends of the helix to assist in the function of the device, or to assist in the ease of use of the device. One example of altering the design to assist the function of the device would be by increasing the envelope or silhouette of the helical retainer to increase its value as a stopper against tissue, or to orient the helical retainer with respect to the tissue in response to suture tension. Other design alterations could be employed to minimize tissue trauma. As will be appreciated by those of skill in the art, this list is not intended to be exhaustive, only illustrative; other purposes also exist, such as joining multiple helical elements for stoppering portions of one or more sutures

A suture may be secured to the helical retainer of this invention in many ways. A particularly useful method of securing a suture to the helical retainer entails a suture leg exiting the tissue, forming a turn or loop passing around two (or more) adjacent coils of the helical retainer and engaging the helical retainer by insertion of the suture into the retainer's lumen at a point between a pair of adjacent coils. Thereafter, the free end of the suture passes between a pair of adjacent coils of the helical retainer within the turn or loop, and in proximity of the loop, and engaging the retainer by insertion of the suture into the retainer's lumen at a point between the coils in such a manner that, when placed under load by tension on the suture exiting the tissue, the adjacent coils are forced together, tightening the grip on the suture. This increased grip, together with any preload and the frictional capstan effect of the suture turns around the coils, very effectively secures the suture against slippage. For purposes of illustration FIGS. 3-6 illustrate a helical retainer 100 and processes of forming exemplar configurations that secure or retain the suture on the helical retainer. The processes shown are for purposes of illustration and, as will be appreciated by those skilled in the art, a wide variety of other steps or suture/retainer configurations for securing a suture using the methods and devices of this invention can be employed without departing from the scope of the invention.

Turning now to FIG. 3A, a helical retainer 300 is depicted in combination with a suture 310. A first end 311 of the suture 310 is connected to the tissue of a patient (as indicated by arrow 312). The second or free end 313 of the suture is wound around or guided against the extension 302 of the helical retainer 300 such that it is fed between a first coil 304 and a second, adjacent coil 304′. This results in the suture 310 being positioned between first coil 304 and second coil 304′ to engage the retainer at that point, as shown in FIG. 3B. The suture 310 is then wrapped around the coils a second time (as shown by arrow 314 in FIG. 3B). This results in the free end 313 of suture 310 being advanced along the coils 304 such that it is positioned at a second engagement point between the second coil 304′ and a third coil 304″, as shown in FIG. 3C. At this juncture it may be desirable, if not already done, to position the helical retainer 300 relative to the tissue being sutured, and to pull on suture leg 313 to tension the suture as desired such that once the final anchoring or restraining tie is in place, and the anchoring element completed, the helical retainer 300 is positioned at the desired location and the suture 310 is properly tensioned, as discussed below.

An extensible element, such as that shown in FIG. 7A or 7B, can be engaged to assist in preventing the suture 310 from feeding between the gap 316 formed between the second coil 304′ and the third coil 304″ which may result from the suture 310 being positioned between those coils. FIG. 3D illustrates the gap 316 which may be formed between the second and third coils (304′, 304″.) The next step in anchor formation involves feeding the suture 310 around and behind the suture leg 311 that is attached to the patient, and threaded between the first coil 304 and a second, adjacent coil 304′ Additionally or alternatively and in the absence of a helical retainer extension, the coils can be configured such that there is a pre-existing gap between the first coil 304 and the second coil 304′ (see FIG. 1C) which would facilitate feeding the suture 310 between the first and second coil, notwithstanding the gap 316 which may be present between the second coil 304′ and third coil 304″. Note that in both FIG. 3A and 3D, the free leg 313 is fed between the first coil 304 and a second, adjacent coil 304′. As will be appreciated, use of the helical retainer 300 does not require the suture section to which it is attached to be a “free” or end leg. The length of suture secured by the helical retainer 300 can also be a length that is engaged at both ends, e.g. by tissue, but which is loose and for which it would be desirable to provide additional tension by anchoring a portion of the loose suture to the retainer. Other suitable applications will be apparent to those of skill in the art based upon the teachings of this disclosure.

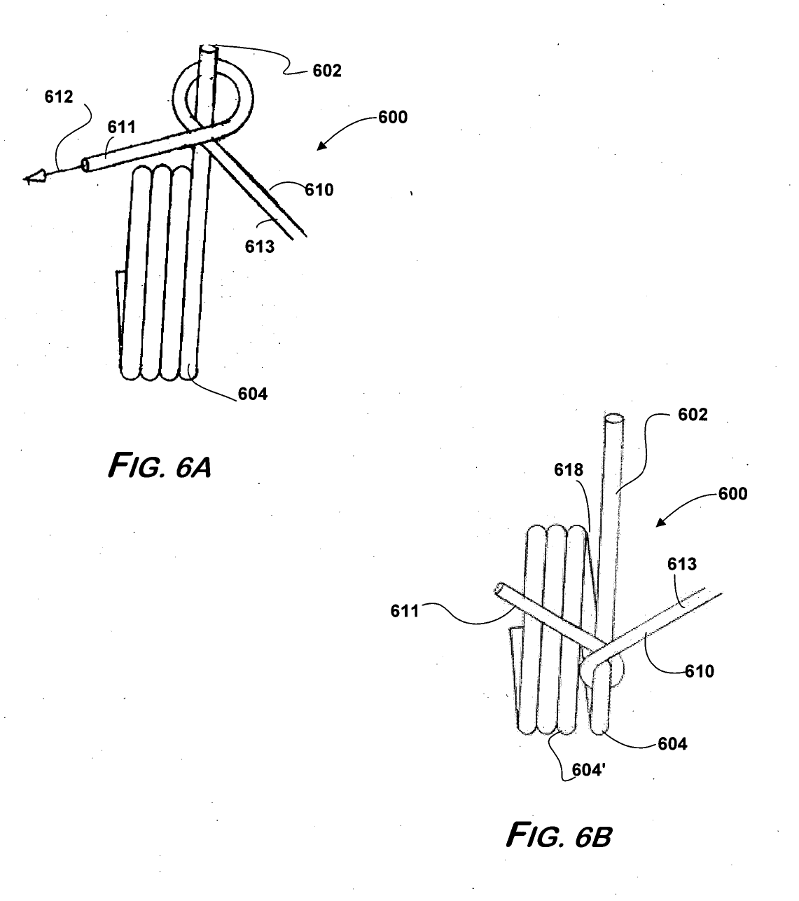

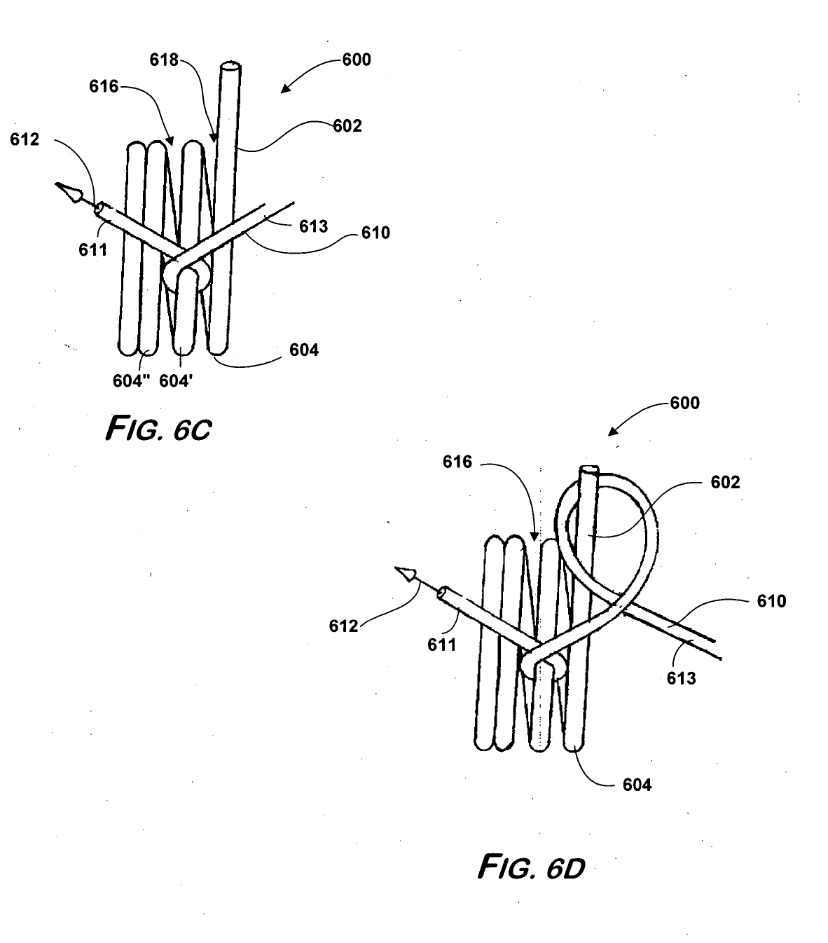

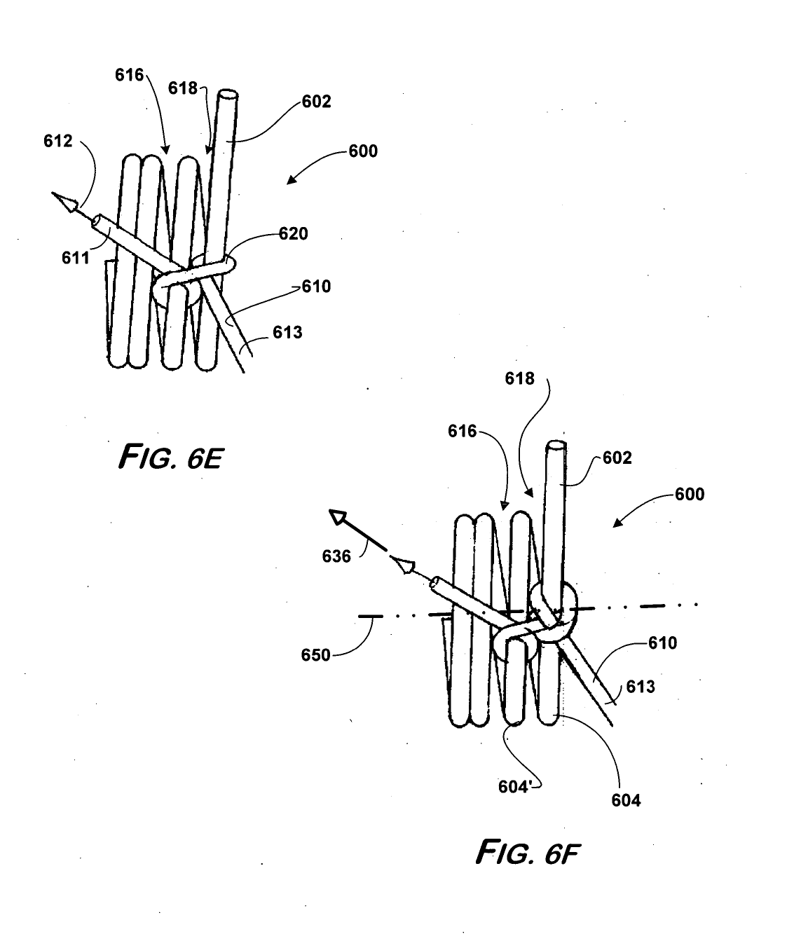

As shown in FIGS. 3D-E the free end 313 of suture 310 is then advanced to lie between coils 304 and 304′ (the end coil and the adjacent coil) in close proximity to the portion of suture 310 between coils 304′ and 304″ in order to lock suture 310 in place. If desired, a security hitch can be added by repeating the step shown in FIG. 3D-E resulting in the configuration shown in FIG. 3F. Rather than repeating the step illustrated in FIG. 3D the security hitch step can be tied in a variety of alternate ways, for example like those of FIG. 5I or 6F, as will be appreciated by those skilled in the art. Once the suture anchoring has been achieved and any optional security steps have been taken as illustrated, tension placed on the first end 311 of the suture 310 is transmitted to pull coils 304 and 304′ of the helical retainer 300 together which clamps the second end 313 of suture 310 in place between those coils, and the holding resistance to tension on suture 310 is increased. This design enables the suture 310 to be anchored using a device without a requirement that the suture 310 be pre-threaded into the device. Nor does this design require that the suture 310 be threaded through an aperture to achieve retention. The anchor formed by this method does not require that the grip on either the helical retainer or the suture be relinquished and reacquired by the surgeon during anchor formation. These are major advantages over conventional knotting and prior art suture retainer use.

Although pre-tensioning the coils such that the coils 304 are positioned adjacent each other into intimate, forceful contact is helpful to hold the suture and maintain the position of the suture on the helical retainer 300 after the tension is set, it will be appreciated by those skilled in the art that the helical retainer 300 will also work similarly even if the coils are not pre-loaded or pre-tensioned, or even if gaps exist between the adjacent coils.

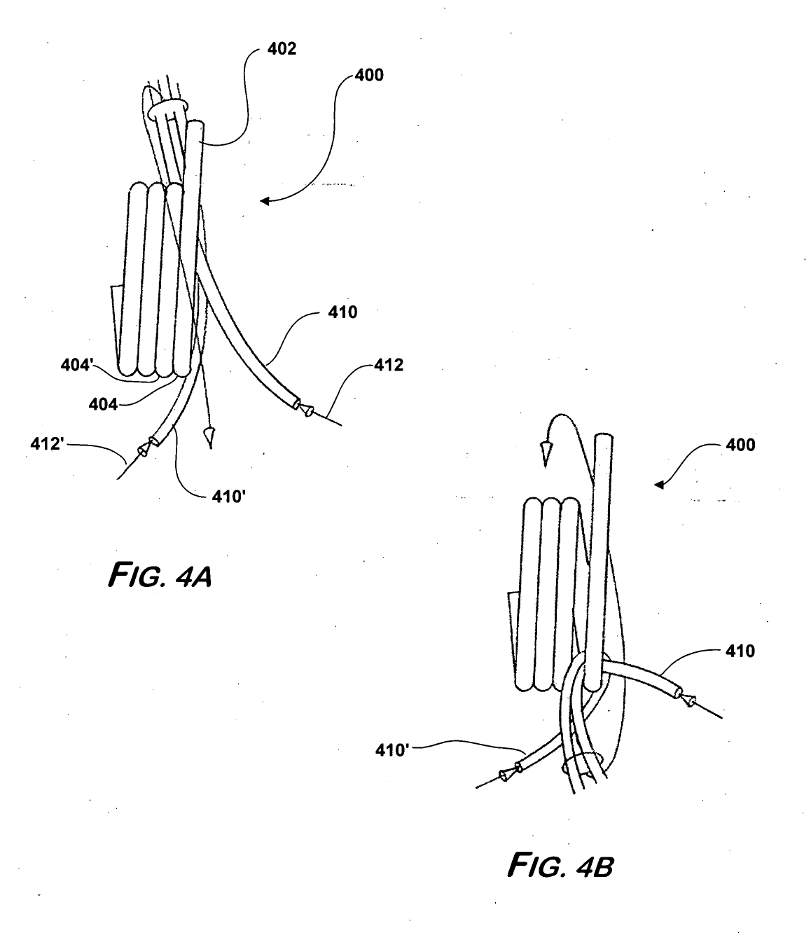

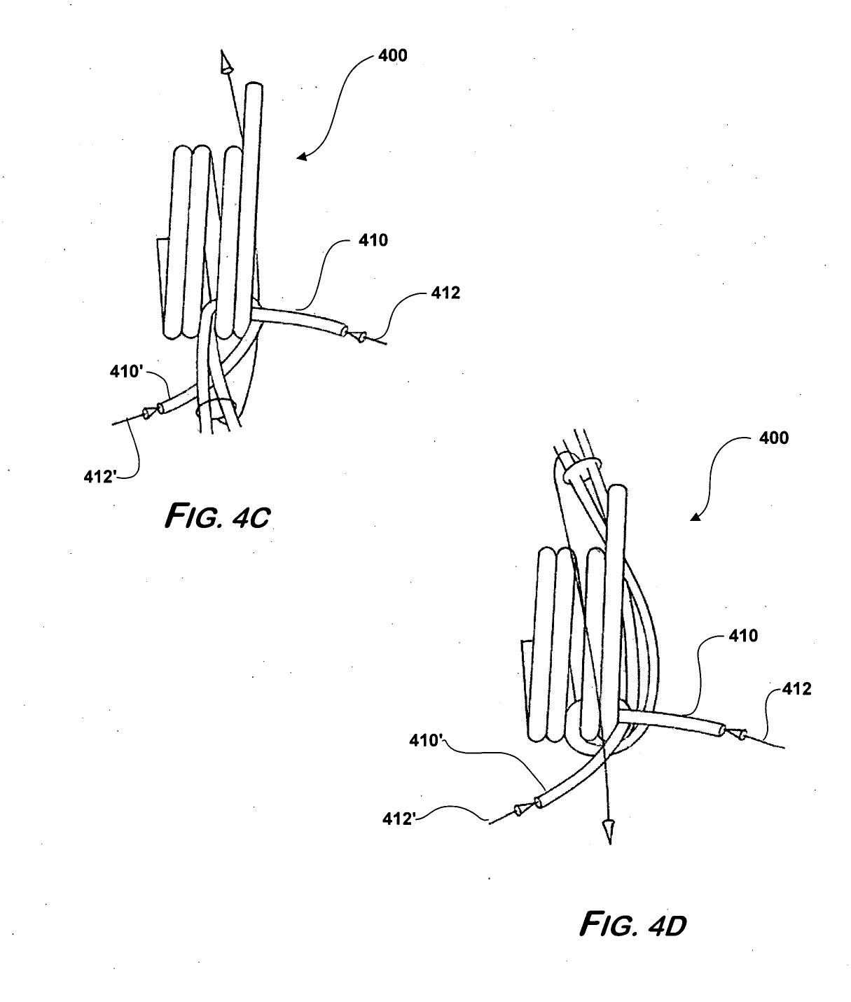

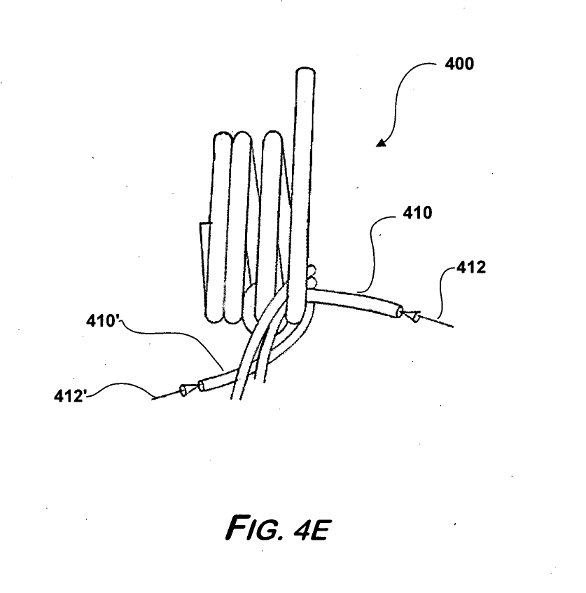

FIGS. 4A-E illustrate a process similar to that shown in FIGS. 3A-E wherein two sutures 410, 410′ are engaged simultaneously. As illustrated, the two sutures 410, 410′ can be received from different locations on the tissue, as indicated by arrows 412, 412′. Alternatively, the two sutures could be received from the same, or substantially, the same area on the tissue.

Turning now to FIG. 5, a helical retainer coil 500 is depicted in combination with a suture 510 as shown in FIG. 5A (which is substantially the same as FIG. 3A). A first end 511 of the suture 510 is connected to the tissue of a patient (in the direction indicated by arrow 512). The second end 513 of the suture is guided against extension 502 of the helical retainer 500 such that it is fed between a first coil 504 and a second, adjacent coil 504′ as indicated by directional arrow 514. As shown in FIG. 5B, the free end 513 of suture 510 is then advanced around the helical retainer 500 a second time (as shown by the directional pull of the suture 514). This results in the suture being advanced along the coils such that it is positioned at an engagement point between the second coil 504′ and a third coil 504″, as shown in FIG. 5C. For purposes of illustration, FIGS. 5D-F illustrate the suture retainer 500 being rotated around an axis 521 as shown by arrow 520. In the first position shown in FIG. 5D the extension 502 is positioned within the illustration proximal and to the right. The suture 510 is looped around two of the coils 504, 504′. In FIG. 5E the helical retainer 500 is has been rotated 520 approximately 150° such that the extension is positioned distally and to the left within the illustration. In FIG. 5F, the helical retainer has been rotated a total of 330° around the axis 521. At this juncture the free end 513 of suture 510 has crossed the portion 511 of the suture leading to tissue. The proximal and distal position as used in this context refers to the position of a portion of the device relative to the remainder of the device as it appears in the drawing. As will be appreciated by those skilled in the art, the proximal and distal positioning of the actual device in situ can change as the device is manipulated and positioned relative to tissue. Finally, with respect to FIG. 5F, the helical retainer coil 500 is rotated 520 another 30°, again around the axis 521 in the same direction which again positions the extension 502 on proximally on the right as shown in FIG. 5G. The helical retainer coil 500 has been rotated 360 degrees and is in the same position as shown in FIG. 5C but the free end 513 of suture 510 is now crossed in front, i.e. proximal relative to the position of the viewer, as opposed to behind the other leg 512 of the suture leading to the tissue as shown in FIG. 3D. The suture 510 is then again guided against extension 502 to lie between the first coil 504 and the second coil 504,′ as shown in FIG. 5H. If desired, as shown in FIG. 5I, the helical retainer can again be rotated and the suture 510 can yet again be guided against extension 502 and fed between coils 504 and 504′ to provide a securing loop which is then advanced such that all suture loops are in close proximity. Additional securing loops can be provided without departing from the scope of the invention. An advantage of this anchoring procedure over that illustrated in FIGS. 3A-F is that by virtue of passing in front of suture leg 511 coming from patient tissue, suture leg 513 may more easily be fed between coils 504 and 504′ rather than into gap 516. Again, in forming this anchor, the helical retainer 500 and suture 510 can be continually grasped and neither need be relinquished during the formation process. Further, as described previously, this anchoring element can be used to terminate two suture legs simultaneously.

Another exemplary anchoring system is shown in FIGS. 6A-6F. With this anchoring system, it is more convenient to grasp and hold the plurality of coils for control of the helical retainer 600 rather than gripping the extension 602. As shown in FIG. 6A, the suture 510 can be looped around the extension 602 such that end 611 of suture extending to the patient (i.e., in the direction of arrow 612) crosses over the free end 613 of the suture. The crossed suture 610 can be advanced along the coils 604, 604′ until the suture 610 is positioned as shown in FIG. 6B. As a result of the positioning of the suture 610, a gap 618 is formed between the first coil 604 and the second coil 604′. Advancing the suture to the position shown in FIG. 6C, another gap 616 is formed between the second coil 604′ and the third coil 604″ as illustrated in FIG. 6C. A second loop can be formed, as shown in FIG. 6D, around the extension 602 in the same sense (or direction) as the first loop and then advanced down the coil as shown in FIG. 6E. An optional securing loop can be formed by repeating the step shown in FIG. 6D, around the extension 602 in the same sense (or direction) as the first loop and then advancing the loop into proximity with the other loops as shown in FIG. 6F.

The resulting anchor shown in FIG. 6E is a securing configuration somewhat in the form of a clove hitch involving two (or more) adjacent coil turns of the helical retainer 600 in which the “crossover” portion of the anchor straddles the coils and the ends of the suture 610 pass between or inside of the outer coils in parallel fashion on opposite sides of the crossover portion. With this anchor, the tension is applied to the helical retainer by a pull 636 more or less transverse to an axis 650 central to the coils. As explained in describing previous exemplary anchors above, this pull again draws the encircled retainer coils together, gripping the suture legs passing between them, increasing resistance to tension.

This anchor, as well as those described previously, is useful when tying one end of a suture, or when tying both ends simultaneously as previously described. A second suture can be applied to the second end of the coil sequentially as well, particularly where a second extension or coil gap is provided in the helical retainer, as shown in FIGS. 1B and D.

As mentioned previously, in performing the steps discussed above, and particularly the steps depicted in FIG. 3D, it may be convenient to provide an extensible element 752 on the distal end of a tool such as a grasper 750. An extensible element is shown as part of a grasper in FIG. 7A. The extensible element 752 can be configured such that it is retractable, as shown by directional arrow 756.

A pair of pincers or jaws 754 working in opposition, are provided on the grasper to hold the coil extension 702 of the helical retainer coil 700. The extensible element 752 provides a guide around which the suture 710 may be passed in order to position the suture 710 between the first coil 704 and the second coil 704′. Without the use of the extensible element 752, it may be difficult to guide the free end 713 of the suture 710 between the first coil 704 and the second coil 704′ to form an anchor. FIG. 7A depicts one possible extensible element as described above incorporated into a grasper. Other extensible elements can be included and/or used in different manners to assist in the use of the helical retainer without departing from the scope of the invention. For instance in FIG. 7B an extensible element 752 is shown which is not part of a grasper. In this illustration the extensible member 752 is placed under the end of the suture 714 leading to the tissue, rather than over as shown in FIG. 7A. In either position it facilitates guiding of the free end 713 of the suture 710 to form an anchor. Once the tool has facilitated guiding the free end of suture it can be retracted of removed from the proximity of the retainer. In general, the tool has an elongated body having a longitudinal body axis; a proximal end having a handle for the user; and a distal end adapted to engage a helical retainer comprising: a grasper formed by a pair of jaws pivoted to work in opposition, and an extensible element extending from the distal end of the tool parallel to the graspers to provide guidance to the suture during engagement with the helical retainer.

As will be appreciated by persons of skill in the art, the tool can alternatively be in the form of a catheter adapted to engage the helical retainer. The structure of catheters is known in the art, including, for example U.S. Pat. No. 5,957,903, and U.S. Pat. No. 6,902,545.

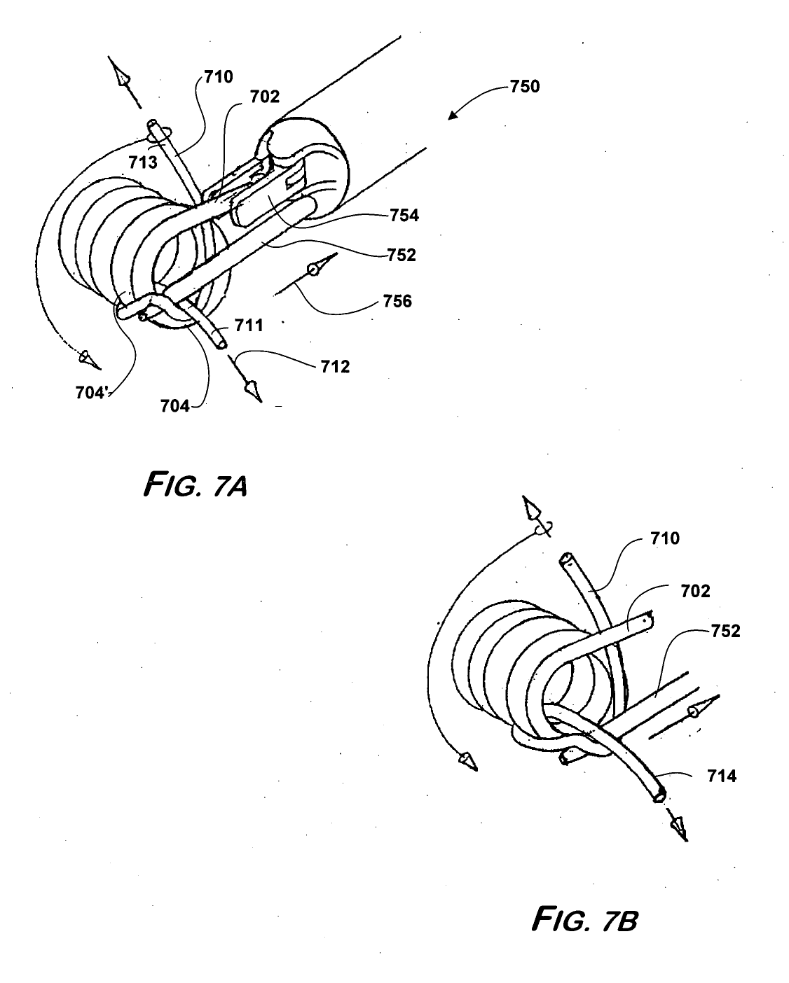

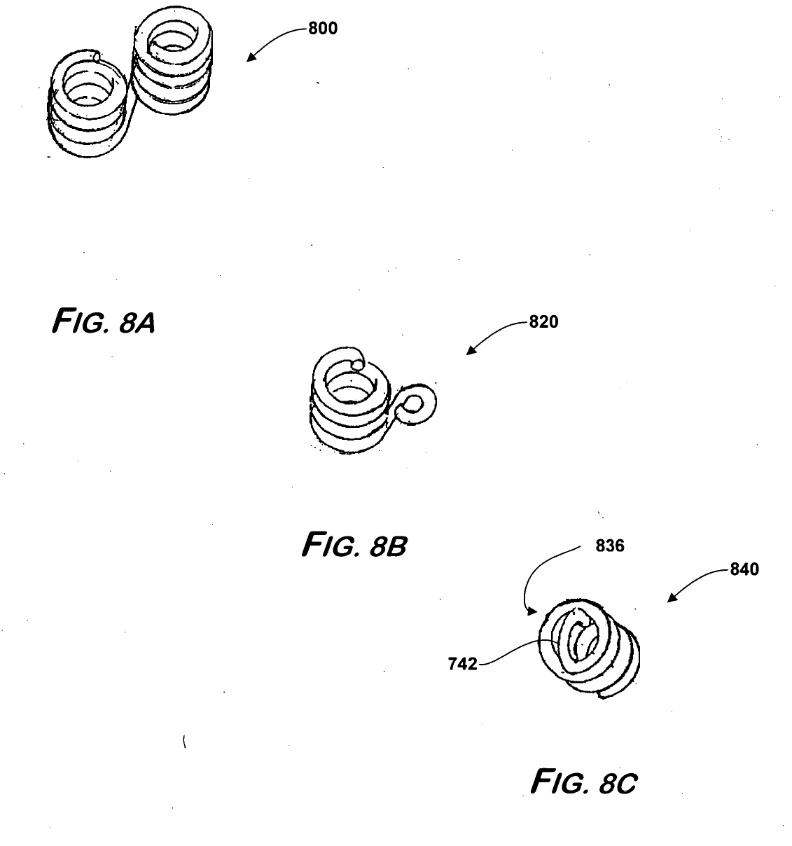

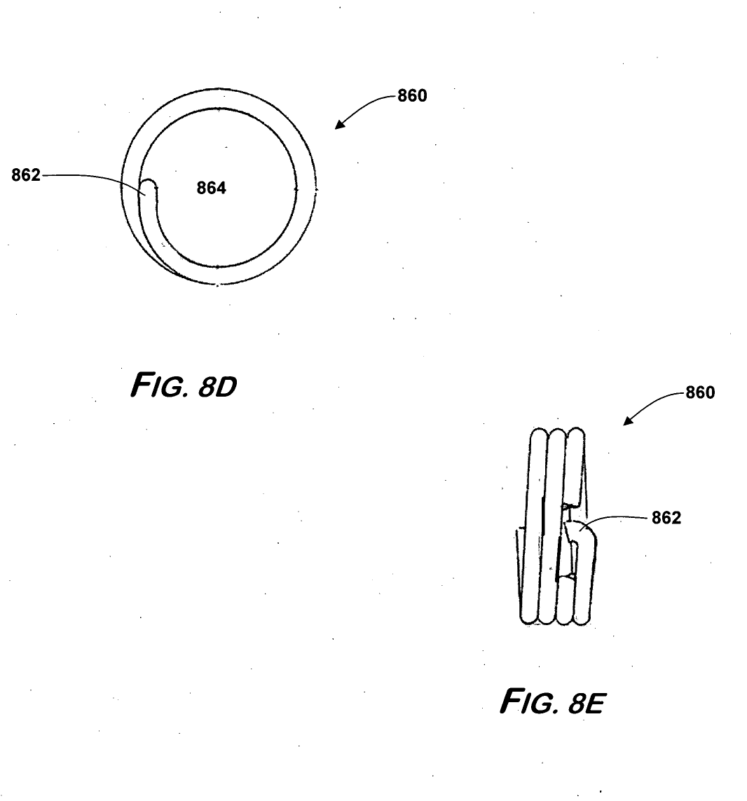

FIGS. 8A-E depict variations in helical retainer 800, 820, 840, 860 designs, including alternate extension configurations that might be useful in various applications. Shape, orientation and placement can be varied to suit the application as can the permutations of features. Some features are directed to facilitating control of the helical retainer by the grasper (e.g., the curled coil extension of FIG. 8B); others are directed to linking two or more sets of coils (FIG. 8A), which can be formed from a single strand or wire, or can be formed by two separate coils providing a mechanism for linking. Still others are fashioned to minimize tissue damage by the extension, i.e., minimizing sharp projections (FIG. 8C), by, for example, turning the coil extension 842 into the center of the coil 834 or curling an extended end into a coil as shown in FIG. 8B. FIGS. 8D-E illustrate an alternative embodiment of a helical retainer 860 wherein the extension 862 is turned into the center of the coil 864 in a manner different than that depicted in FIG. 8C. Other variations within the scope of the invention can be included without departing from the scope of the invention, including other elements or molded attachments that can be included with the helical retainer, either for the purposes noted above, or for other purposes. As presented, all sketches assume the use of a left handed helical retainer (such as shown in FIG. 2A). However, as will be appreciated by those of skill in the art, it is equally acceptable and within the scope of the invention to use right handed devices (such as that shown in FIG. 2B) with appropriate inversions of the procedures described herein.

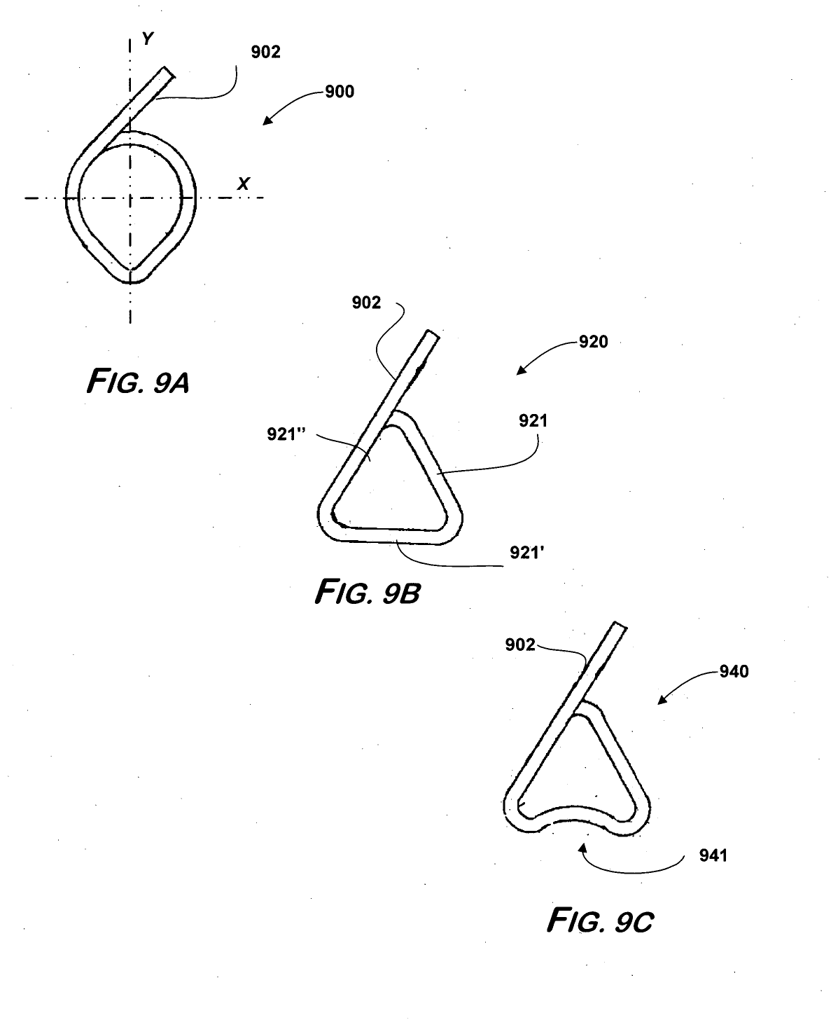

In the helical retainers described, the profile or lumen shape formed by the inner circumference of the coils needs not be round, nor does the axis need be straight. FIG. 9 depicts coil shapes which may have utility, either to locate the knots relative to the extension or to tissue, to facilitate tensioning, or to serve other purposes. FIG. 9A depicts a helical retainer 900 that, from the top end, is tear-shaped or egg-shaped in its inner geometry. In such a configuration, the x axis has a length less than its y axis, when measuring an x and y axis that intersect in the center of the retainer coil 900. FIG. 9B depicts a helical retainer coil 920 that, from the top end, is essentially triangular, i.e. an inner geometry formed by three intersecting lines, each of which can have the same length 921, 921′, 921″. FIG. 9C depicts a helical retainer 940 that, from the top end, is substantially triangular, with one side of the triangle indented 941 to form a concave exterior surface. As will be appreciated by those of skill in the art, these geometries can be used in various orientations or in different combinations of features. Also, other geometries can be employed without departing from the scope of the invention. Similarly, the location, angle of departure point of the extension (i.e. where it departs from the profile shape of the helical retainer 900) can change, without departing from the scope of the invention.

FIG. 10 illustrates a situation where a helical retainer 1000 is used to terminate different portions of a suture, or different sutures 1010, 1010′, in this instance using the anchoring arrangements illustrated FIGS. 3A-E to form anchors 1030, 1030′ on either end of the helical retainer 1000. The sutures 1010, 1010′ can, as illustrated, be received from two different directions as illustrated by directional arrows 1012, 1012′.

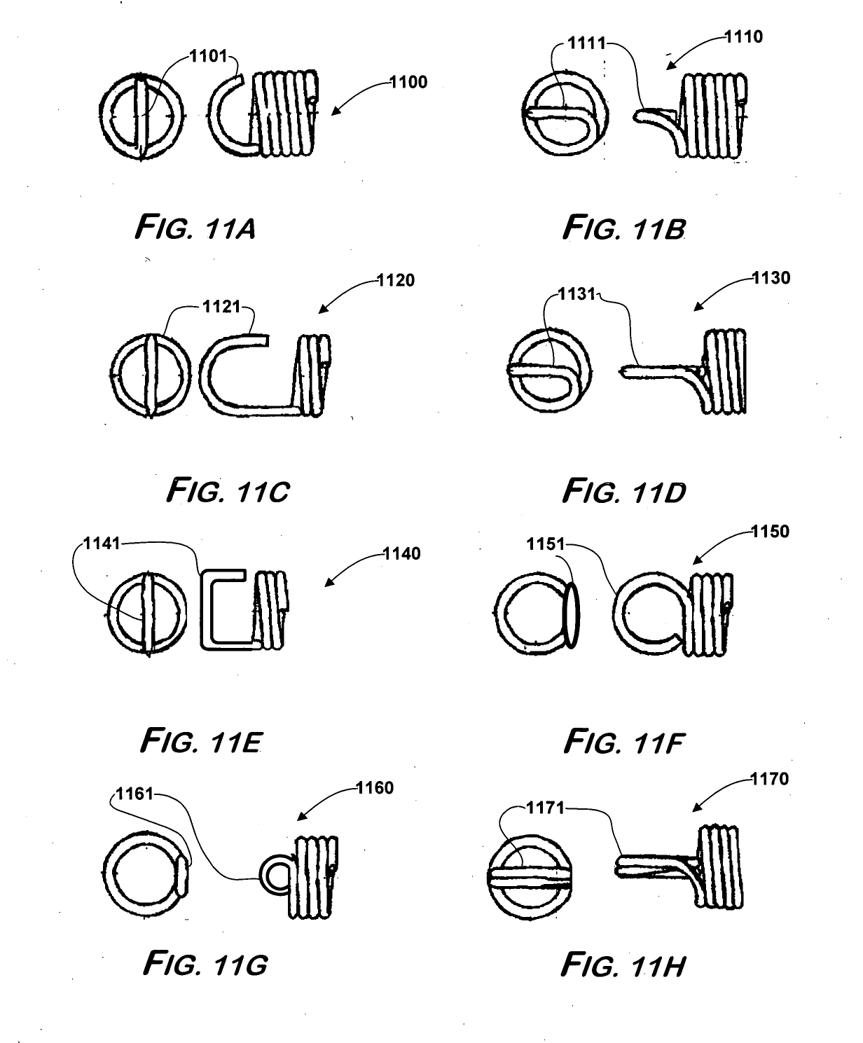

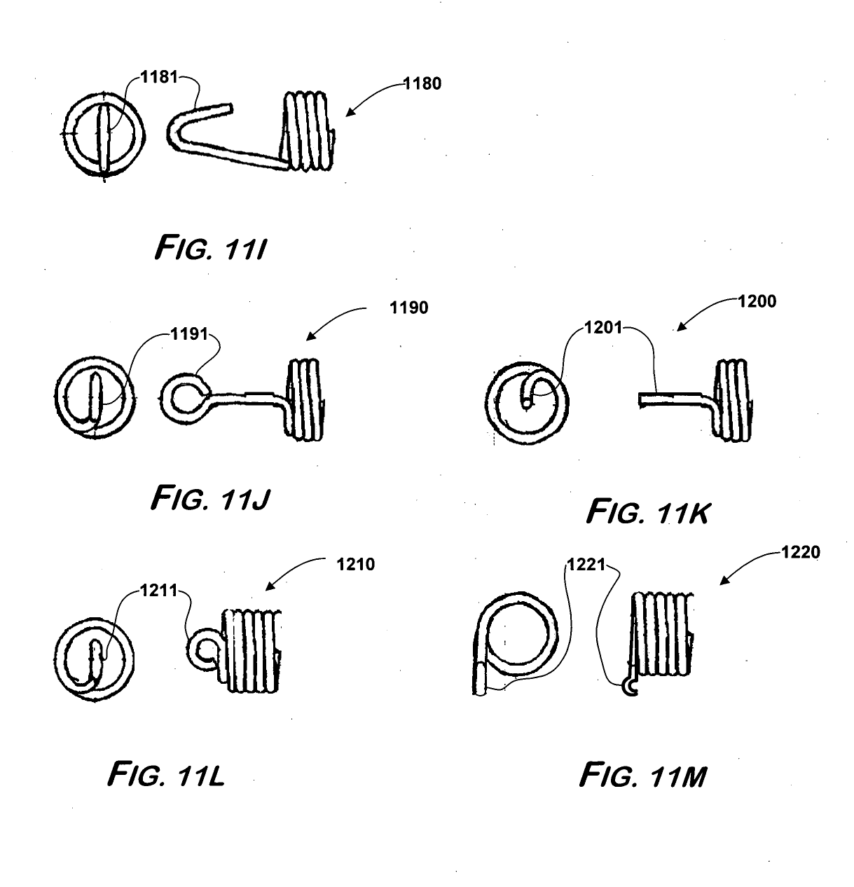

FIGS. 11A-M depicts a top view and a side view of a variety of extensions suitable for use on any of the coils depicted herein. The retainers can be made from round or shaped wire, for example, square wire, and the coils are usually close-wound and in contact with each other. FIG. 11A is an example of a helical extension 1100 in the form of a half-loop extending along the longitudinal axis with an opening 1101. FIG. 11B is an example helical retainer 1110 with a extension having a more gradually curved departing end 1111 from the coiled body which provides more of a gap 1112 for guiding the suture. FIG. 11C depicts a helical retainer 1120 having an end featuring a raised hook 1121 providing a larger surface to grasp. FIG. 11D illustrates a helical retainer 1130 having an end with a raised hook 1131 which has a more gradually curved departure from the coiled body. FIG. 11E illustrates a helical retainer 1140 having an ending with a rectangular hook 1141. FIG. 11F illustrates a helical retainer 1150 having an end configuration with a full loop 1151 that is aligned with the outer diameter of the coils. FIG. 11G illustrates yet another helical retainer 1160 having a reduced diameter side loop 1161. FIG. 11H illustrates another helical retainer 1170 having a double twisted loop 1171. FIG. 11I illustrates a helical retainer 1180 with an alternative end configuration featuring a v hook 1181 over center. FIG. 11J illustrates yet another helical retainer 1190 having an extended loop 1191 end. FIG. 11K illustrates a helical retainer 1200 with a straight end from the center 1201. FIG. 11L illustrates a helical retainer 1210 having a reduced diameter center loop configuration 1211. FIG. 11M illustrates yet another helical retainer 1220 having an offset hook 1221.

In embodiments of the invention in which the helical suture retainer is to be used for surgical or other medical applications, the suture retainer should be sterilized, and any kits containing the helical retainer and any attached sutures should be sterile.

What is claimed is:

1. A helical retainer comprising:
a sterile coiled body adapted and configured to secure a surgical suture, the coiled body comprising a plurality of coils and being formed from a strand; and
an extension of the strand extending from the coiled body adapted to engage a suture or to be grasped by a holding tool.

2. The helical retainer according to claim 1 wherein the extension is coiled.

3. The helical retainer according to claim 1 wherein the extension extends tangentially from the coiled body.

4. The helical retainer according to claim 1 wherein the plurality of coils of the coiled body form a lumen and further wherein a first end of the strand is terminated within the lumen.

5. The helical retainer according to claim 1 further comprising a second coiled body joined to the first coiled body.

6. The helical retainer according to claim 1 wherein the coils are pre-tensioned.

7. The helical retainer according to claim 1 wherein the helical retainer is preloaded with a suture.

8. The helical retainer according to claim 1 wherein the helical coil is further adapted to permit a secured suture to be disengaged from the helical retainer without damaging the suture.

9. The helical retainer according to claim 1 further comprising a gap between adjacent coils.

10. The helical retainer according to claim 1 further comprising a plurality of gaps between pairs of adjacent coils.

11. The helical retainer according to claim 1 wherein the coiled body is further adapted to be engaged by a surgical suture between at least two pairs of adjacent coils to secure the surgical suture.

12. A helical retainer comprising:
a sterile coiled body adapted and configured to secure a surgical suture, the coiled body comprising a plurality of coils; and
a gap between two adjacent coils.

13. The helical retainer according to claim 12 wherein the helical coil is further adapted to permit a secured suture to be disengaged from the helical retainer without damaging the suture.

14. The helical retainer according to claim 12 wherein the coiled body is further adapted to be engaged by a surgical suture between at least two pairs of adjacent coils to secure the surgical suture.

15. A method of using a helical retainer comprising:
placing a helical retainer at a desired location, the helical retainer comprising a plurality of coils;
engaging a suture with the helical retainer at a first engagement point; and
engaging the suture with the helical retainer at a second engagement point to secure the suture to the helical retainer.

16. The method of claim 15 wherein engaging the suture at the first engagement point comprises engaging the suture at the second engagement point and advancing the suture along the helical element from the second engagement point toward the first engagement point.

17. The method of claim 15 wherein the first and second engagement points are between adjacent pairs of coils.

18. The method of claim 15 further comprising releasing the suture from the helical retainer.

19. The method of claim 15 further comprising: applying tension to the suture.

20. The method of claim 19 wherein applying tension comprises applying tension to move one coil of the helical retainer toward another coil of the helical retainer to more tightly engage the suture between the coils.

21. The method of claim 15 further comprising: engaging the suture with the helical retainer a second time at the second engagement point.

22. A kit for suturing comprising:
a sterile coiled body adapted and configured to secure a surgical suture, the coiled body comprising a plurality of coils and being formed from a strand and an extension of the strand extending from the coiled body adapted to engage a suture or to be grasped by a holding tool; and
a suture secured by the helical body.

23. A tool adapted to engage a suture with a helical retainer comprising:
a proximal end having a handle; and
a distal end adapted to guide a suture into engagement with the helical retainer.

24. The tool according to claim 23 wherein the distal end is retractable with respect to the proximal end.
